SEQUENCE LISTING (16 sequences)

SEQ ID NO: 1 - DNA, 2190 bp, Oryza sativa
  misc_feature: WAK91
atgatgaccatcattcaaccaccggctatggcgatggcgatggcgatggcgctgctgctgctgctgcttctgcagctgtggtcggtggaagcgcaggtcgcagcgccgccgccggcgagctgcccggacaggtgcggcgacgtgagcgtgccgtacccgttcggcatccgcgacggctgccacctcccgggcttccgcctcacctgcgacgccacccacaccccgccgcgcctgatgctcggcaacggcaccctccaggtcgtcgacatctccctggccaactccaccgtgcgcgccctcgacctcgccggcgccgtcaacttcacctacgacgtgtcgaagctcgcccccagcggcagcggcacgtggtccagcctcggcaccgtcgccggcgccggcccgtacgtcgtctccgagcagcgcaaccggctcgtcgtcacgggctgcaacgtccaggccacgctcgccggggagaacaccaacatcatcggcggctgctcctccttctgcccggtcagcgagatgttcaccagcgtggcggcgaccgtccccgtcgtccccggcgccggcgccgacaacgccaccgatggcggcttcatctgctccggcaccagctgctgcgagacgcccatcgccatcggccgcccctcctacctcgtgcagttcctcagccttgaccagaaccaggagctcaccggcaagctccccgtcgccgtgcgcatcgccgagcgtgggtggttcgagggcgtcgccggcgagctgctcaacaccagctccgactccgccgccgccctccggacgccggtcccggtggtgctggagtgggtggtgtcgccgacgctggaggcggtgctgcagggcgtcaccgggcagttcgccgacgaccgcaactggtcgtgccccgcggacgcggcgaggagcgcatgccggagcagcaacagcttctgcagcaacgtcaccggcaactaccgccgcggctacgtgtgcaggtgccggcgaggctacggcgggaacccctacgtcgccggcggatgccaagacatcgacgagtgcaagctcgccgggaggtgctacggcgagtgcaccaacacgccgggagattaccagtgccggtgcccgcgcggcgctcgtggcgacccgcgcattccaaacggctgcgtcaaaactaatctaggtttaagtgttggtattggagttggcagtggagctgggcttttggtaatgggactgggcgctgcctttttaaaacgtaaggttaagaaacagagagcaagaatgctgaggcagaagttcttcaagcagaaccgaggtcatttgttgcaacaactagtgtctcagaaggctgacattgctgaaaggatgatcatccccttgtcagaactggagaaggccacaaacaattttgataaatcacgtgagcttggaggaggggggcacggcaccgtgtacaaagggattttatcggatcttcatgttgtcgcgatcaagaaatctaaggaggcagttcaaagagagattgacgagttcataaatgaggtagccattctctcgcaaatcaaccatcgtaatgtggtcaaactttttggatgttgtcttgagacagaagtgccattgctggtttatgaattcatatcaaatggtaccctttaccaccatcttcatgttgaaggaccaatgtcattgccatgggaagataggctcagaattgcaaccgaaaccgctagagcacttgcctaccttcactcagctgtgtcgttccctataatccacagggatatcaaatcccataacatcctattagatggttcactaacaacaaaagtgtccaattttggagcttcaaggtgcattccagctgaacaaactgggataacaaccgttgttcaaggaacactaggatatctggaccccatgtactactacacagggcgtctcactgagaaaagcgatgttttcagcttcggcgtcgttctaatagagttgcttactaggaagaaaccatactcatacagatcgcctgatgatgaaagtcttgtaacacatttcactgccctactcacacaaggcaacttgggcgacatacttgatcctcaggtcaaggaagagggaggcgaagaagttaaagagatagctgtgttagctgtggcatgtgccaagctgaaagtagaggagcggcctactatgaggtag

SEQ ID NO: 2 - protein, 729 aa, Oryza sativa
  MISC_FEATURE: WAK91
MMTIIQPPAMAMAMAMALLLLLLLQLWSVEAQVAAPPPASCPDRCGDVSVPYPFGIRDGCHLPGFRLTCDATHTPPRLMLGNGTLQVVDISLANSTVRALDLAGAVNFTYDVSKLAPSGSGTWSSLGTVAGAGPYVVSEQRNRLVVTGCNVQATLAGENTNIIGGCSSFCPVSEMFTSVAATVPVVPGAGADNATDGGFICSGTSCCETPIAIGRPSYLVQFLSLDQNQELTGKLPVAVRIAERGWFEGVAGELLNTSSDSAAALRTPVPVVLEWVVSPTLEAVLQGVTGQFADDRNWSCPADAARSACRSSNSFCSNVTGNYRRGYVCRCRRGYGGNPYVAGGCQDIDECKLAGRCYGECTNTPGDYQCRCPRGARGDPRIPNGCVKTNLGLSVGIGVGSGAGLLVMGLGAAFLKRKVKKQRARMLRQKFFKQNRGHLLQQLVSQKADIAERMIIPLSELEKATNNFDKSRELGGGGHGTVYKGILSDLHVVAIKKSKEAVQREIDEFINEVAILSQINHRNVVKLFGCCLETEVPLLVYEFISNGTLYHHLHVEGPMSLPWEDRLRIATETARALAYLHSAVSFPIIHRDIKSHNILLDGSLTTKVSNFGASRCIPAEQTGITTVVQGTLGYLDPMYYYTGRLTEKSDVFSFGVVLIELLTRKKPYSYRSPDDESLVTHFTALLTQGNLGDILDPQVKEEGGEEVKEIAVLAVACAKLKVEERPTMR

SEQ ID NO: 3 - DNA, 2354 bp, wheat
  misc_feature: TaWAK91
gcggtgttgctcctgcagctctgcctcccaatggcggcggctcaggttaccccaggcgcagggacgccgccgccgcgctgcccgaccagctgcggcggcgtgaaagtgccgtacccattcggcatcgggaacggatgccaccggccgggattcaacctcacctgtgaccggacgcgcggcagagagccgcggttgctcgtcggcagcgacctccaggtcgtggagatctctctggccaactccacggtgagaatcttggacagggccggccaggtcaagctcaccttctccgagggcctcgatggcaacgggacatggggcggcctcggcgccggcggcccttacgtgctctcggagatgcgcaaccacttcgtcgttacggggtgcaacgtgcaggccacgctggtcgggaacggcggggtcgtaggctgctgctcctccttctgctccatcaacgacaagtggacgggcgtcgtgacgagctccgctggctacggcggcgccgcctgctctggcatcggctgctgcgagacgcccatccccataggccgcccttcctacgacgtggagatgaggtcgctggatgcgagcaacgtgtacgccgacaggctgcccatcgcggtgcgcatcgccgagagagggtggttcgaaggcgcctccaccacgctcctcaacgactcaaccgggtactcgccctcccgccagccggctgtgcccgtcgtcctcgactttgcggtggattccaagccggtggtgctgccaggggtggcaacgtcgggctgccctgtggacgcacgaaggagcgcgtgccagagcagccatgcttcctgccataacgtctccggcatgtaccgcagcggctacgtgtgccggtgcctagacgggtaccaaggcaacccatacctcacaggcggatgccaagacatcgatgagtgcgcgcttccgggcatgtgcttcggtgagtgcaccaacacggccggaggacacctatgccggtgcccgcgtggcgcccaaggtgacccgctcataagaaatggctgcataaaatcttctctaggtgagcaccaaaaccaataatacatgcagatcggagcacagttcatgctccgttttagatgtcagagattcttccttcattacaaagacacattttctgtgcacattcttcaggtttaagtgtgggcataggagttggcagtggggccagccttctactcatggtacttggtgctattttggtgtcccgaaagatgaagcaacgaagggcagaaatgctcaagaagagattcttcaggcaaaatcgggggcatctgttgcaacaattggtgtctcagaaggcggacattgccgagaggatgatcgtccccttggtggaactacaaaaggccacaaacagtttcgacaaagctcgtgagatcggtggaggagggcacggaacggtttacaaaggcatcatgtcagacctgcatgtcgtggcgatcaagaagtccaaggtcacgattcagagggagattgacgagttcataaatgaggtggccatcctctcgcagatcaaccaccgaaacgtggtgaagctctttgggtgctgccttgagacggaggtgccgctgctggtgtacgagttcatctccaatgggaccctctaccatcatcttcatgtccaggaaccagcgccatccctaacatgggaagataggctaaggaccgcaaccgaaactgcgagagctctcggctaccttcactcggcggtgtcctttcctatagtccacagggatatcaagtcccaaaacattctgttagatggcagtctcatagcaaaggtgtcagactttggagcttcgaggtgcattcaagtcgatcaaacagagaccgcaaccgctatccaagggacatttgggtacctggacccattgtacttctactcgggacagctcaccgagaagagcgacgtttacagcttcggcgtcctcctcatggagcttctcactaggaaaaaaccatgctcatatcggtcatccgaggaggaagcccttgtcacatactttgccgcttcgctagcagcagacaagctggttcgcgtgctagatcctcaggtcgtgaaagaaggcggcaaggaggttgaagaagtagctgtgttggcagtggcatgtgtgaggattgaagtagaccagcggccaaccatgaggcaagtggagatgacacttgagaaccttgggggctcgcatgatagctttgtaatgcatgacctggatgcgcccaagtatccaatgattgagggtacgaacatggaggaaacaagccagcagtatagctatgaagcagagtatttgttgtcatcaaggtacccccggtag

SEQ ID NO: 4 - DNA, 2361 bp, sorghum
  misc_feature: WAK91-like
atgccacaactactcgaaacagcaatagacgacatttctggttctgcaggcggcgtggtgatggttccggcagcggcgctgatgctagttctgcagctcttttccttggcttcagcagctcagtccggaaacgtaggcccgccgccgacctgcccatccagctgcggcaacctgagcgtgccgtatccattcggcatcggcgccagctgctccctccctggcttcaacctcacctgcgaccgaacgcgccacccgccgcggctgctggtcggagatggcgccctccagatcgtggagatctctctggccaactccacggtccgggccctgtacacagcgggcgccgttaacatcaacttcagcacatcggccacatccgtcgacggcagcggcacctggagcggcggcctcggcgctgccagtgacagcccgtacgtggtctcagagtggcgcaaccagttggttgtcacagggtgcaacgttcaggggacgctgctcgggggcagcggcaacgtcatcaccggctgctcctccttctgctccatcgacgacaagtggaccggcgccgtggtaaccacccctgacgagggcgccaccgcgtgctccggcatccgctgctgcgagacgcccatccccattggccgcccgtcctacaacgtccagttcaagtacctggacgttgagaacacgggtttgctacctgtggcagtgcgcatcgccgagcatggttggtttgacagtgtcgccgcgcaaatgctgaacaactcagtgagggattccacgctcgggacgccggttccagtggtactggactgggcggtggcgtcgaccccaatagtcccgggaacgcctgctgcggatgccgccggcaactcgtcctgcccctcggacgcggcgaggagcgcctgccgcggcagccacagcgcctgccataacgtcaccaacaactaccggactggctacgtgtgccgctgccagaacggctacgacggcaacccctacctgtccagcggatgccaagatatcaatgagtgcgaagtcccggggaattgtttcggtatatgtacaaacacggacggatcgtacgagtgccggtgcccacgcggtgctagtggtaacccatacgtggaacatggctgcatcaaatcttctctagggctgagtattggccttggagttggcagcggggcaggtcttttggtgctggtactcggtgctgtctttgtgactcgtaggattaagcatcgcagggcaagaatgctgaagcagaagttcttcaagcagaaccgtggacatttgttgcaacaattggtgtctcaaaaggctgatatcgctgagaagatgatcatccccttgatagagctggaaaaagccacaaataattttgacaaagctcgtgagcttggcggaggtggacatggtactgtctacaagggcattctatctgaccagcatgttgtcgcaataaaaaagtcaaaagtggcaatccaaagagagattgatgagtttattaatgaggtagctattctctctcaaatcaatcatcgaaatgtcgtaaaactctttggatgttgccttgagacacaagtgccactattggtttacgaattcattccaaatggtactctttatgatcatcttcatgttgaaggaccagcaacattatcatgggagtgtaggctgagaattgcaacggaaaccgctagagcactagcctaccttcacatggctgtgtcattccctataatccacagagatatcaagtcccataatattcttttagatggctcaatgatagcaaaagtgtccgatttcggagcttcaagatgcattcccacgtacaacactgggatttcaactgctatccaaggaacatttggatacttagatcctatgtactactacactggaagactcactgagaagagtgatgtttttagctttggcgttgttcttatagagctgctgactaggaaaaagccctactcctatagatcacctaaggatgatggtcttgttgcacatttcacggcattgctctcggaaggcaatttggtccatgtacttgatcctcaggtcatagaagaggcaggtgaacaagtgggggaagtagctgcaatagcagcatcatgtgtcaaaatgaaagcagaggatcgaccgaccatgagacaggtggagatgacccttgaaagcattcaagcacccgtacaaaacgttgtgcaacgtacaggtacaaggatatgtgatgagaaacagaaggctgtcatgtatccattagtggagggtacaagcaagcaggagtcaagtagacagtatagtcttgaagaagagtacctcttgtcagcaagatacccgcgatag

SEQ ID NO: 5 - protein, 786 aa, sorghum
  MISC_FEATURE: WAK91-like
MPQLLETAIDDISGSAGGVVMVPAAALMLVLQLFSLASAAQSGNVGPPPTCPSSCGNLSVPYPFGIGASCSLPGFNLTCDRTRHPPRLLVGDGALQIVEISLANSTVRALYTAGAVNINFSTSATSVDGSGTWSGGLGAASDSPYVVSEWRNQLVVTGCNVQGTLLGGSGNVITGCSSFCSIDDKWTGAVVTTPDEGATACSGIRCCETPIPIGRPSYNVQFKYLDVENTGLLPVAVRIAEHGWFDSVAAQMLNNSVRDSTLGTPVPVVLDWAVASTPIVPGTPAADAAGNSSCPSDAARSACRGSHSACHNVTNNYRTGYVCRCQNGYDGNPYLSSGCQDINECEVPGNCFGICTNTDGSYECRCPRGASGNPYVEHGCIKSSLGLSIGLGVGSGAGLLVLVLGAVFVTRRIKHRRARMLKQKFFKQNRGHLLQQLVSQKADIAEKMIIPLIELEKATNNFDKARELGGGGHGTVYKGILSDQHVVAIKKSKVAIQREIDEFINEVAILSQINHRNVVKLFGCCLETQVPLLVYEFIPNGTLYDHLHVEGPATLSWECRLRIATETARALAYLHMAVSFPIIHRDIKSHNILLDGSMIAKVSDFGASRCIPTYNTGISTAIQGTFGYLDPMYYYTGRLTEKSDVFSFGVVLIELLTRKKPYSYRSPKDDGLVAHFTALLSEGNLVHVLDPQVIEEAGEQVGEVAAIAASCVKMKAEDRPTMRQVEMTLESIQAPVQNVVQRTGTRICDEKQKAVMYPLVEGTSKQESSRQYSLEEEYLLSARYPR

SEQ ID NO: 6 - DNA, 1501 bp, Oryza sativa
  misc_feature: promoter OsWAK91
accgactatgagacaggtggaaatgacactcgagagcattcgatcatcatcactgcagcaagaagtattgcatagtgttagcacaaagaaatctaaggagcttcatgtctcatggagccatgcaataagtgagggtacaagcttagattcaactaggcaatatagtctcgaagaagagaacttgttatcatcaaggtatcctcgatagcgattttttaatttttcttgtgtgagtttggatcgatatagaaagcttatgttgtcctcgatgtacaaggtctccctagtttttttttctctccatgtatgatacgcacgtagtatttcatagctaaatgtagtaatatcttattttattgttcttcatctgaatgtgtatttttctgtttgatttcatccaagtaaatatcaagataccttttgtattgtactaactatggtttcgggctaccaaacgacagctgcttcgttcgttgatgttcgcaactaatcttttgcgtacgcaaaacgaaacgactcattagcatatttttaattaaatattagttcatttttaatatgattgtttaaaacaagttttgtgtaatttactttatttagtagtttaaaaagcttgtacacgaaaagttgtcgacagttcagcgatgatggttccagcgcccagcggcaacttgtgcggcggcgctgtcatctgcagaaacagatttccggggacacaatgccgccagcgatgatctgtagaaacggtcaaccatccagtccaatccacacctacctgatcgatcggtcaacgcggatacaacaacgcagcatatgcatgtgcggctttggatttgtcaagcaaagcgccccacgtcgtcagtgccgccagtttggactaaattttgccttactaaattttggtattacttaattttgttatagttactaaaattttggcaacttgtcacaattttgttaggatttcttatatagtcactaaaatttagcagcaaactaaatgtagccactttttttagcaacattaccaaaatttggtaagattgaaaatgacatcaaagtgaacatgcccttagttccaaaccaattttttttggcactgtcacatccaatatttgacacatgcgtgaagtattaaatatagaaaaaaacaattatacagttcgtcagacgaatcttttaagtttaattgtgccatgatttaacaatatgatgctacagtaaatatttactaataacggattaattatacttaataaatttgtctcacagtttttggtggaatatataatttattttattattagactatataaatatgtgtccatatctaatgtaatatacagtatatgggtaaatttttttgccataggcacgccaaattgcccatacctcagtgtatgtgtactacttatatcgacgctctgtctggccatatattaggaatataaacacagactatgttccagcgatgatgaccatcattcaaccaccggctatg

SEQ ID NO: 7 - DNA, 49 bp, artificial sequence
  primer ATFL14-F
ggggacaagtttgtacaaaaaagcaggcttcattcaaccaccggctatg

SEQ ID NO: 8 - DNA, 49 bp, artificial sequence
  primer ATFL14-R
ggggaccactttgtacaagaaagctgggtccacaccatgctcttgctgc

SEQ ID NO: 9 - DNA, 31 bp, artificial sequence
  primer ATp4-F
atctaggatccaccgactatgagacaggtgg

SEQ ID NO: 10 - DNA, 31 bp, artificial sequence
  primer ATp4-R
agtacgaattccatagccggtggttgaatga

SEQ ID NO: 11 - DNA, 24 bp, artificial sequence
  primer Tail6
gtactgtatagttggcccatgtcc

SEQ ID NO: 12 - DNA, 20 bp, artificial sequence
  primer AT23F
gcaccaactgctctgtttca

SEQ ID NO: 13 - DNA, 20 bp, artificial sequence
  primer AT23R
gcaatggcacttctgtctca

SEQ ID NO: 14 - DNA, 22 bp, artificial sequence
  primer ATq41F
ttgcaagcatgacagcggttac

SEQ ID NO: 15 - DNA, 22 bp, artificial sequence
  primer ATq41R
aaccctcttcaaggccaaacgg

SEQ ID NO: 16 - DNA, 2190 bp, artificial sequence
  Optimized sequence of OsWAK91
atgatgaccatcatccaaccaccagcgatggcgatggcgatggcgatggccctcctcctcctgctcctgctccaactctggtccgtggaggctcaggtcgctgctccaccaccagcgtcctgcccagaccgctgcggcgacgtgagcgtcccgtacccattcggcatcagggacggctgccacctgccaggcttcaggctcacctgcgacgctacccataccccaccaaggctgatgctcggcaacggcaccctgcaggtggtcgacatctccctcgccaacagcaccgtgcgggcgctggacctcgctggcgctgtgaacttcacctacgacgtctccaagctggccccctccggcagcggcacctggtccagcctcggcaccgtcgctggcgctggcccatacgtggtcagcgagcaacgcaaccggctggtggtcaccggctgcaacgtgcaggctaccctcgcgggcgagaacaccaacatcatcggcggctgctccagcttctgcccagtctccgagatgttcaccagcgtggccgcgaccgtccccgtggtcccaggcgctggcgctgacaacgctaccgacggcggcttcatctgctccggcaccagctgctgcgagaccccaatcgctatcggcaggccatcctacctcgtgcaattcctgagcctcgaccaaaaccaggagctgaccggcaagctcccagtggccgtccgcatcgctgagaggggctggttcgagggcgtggctggcgagctgctcaacacctccagcgactccgctgctgccctgcgcaccccagtcccggtggtgctggagtgggtggtcagcccaaccctggaggctgtgctccaaggcgtcaccggccagttcgctgacgacaggaactggtcctgcccagctgacgctgctaggagcgcttgccgctccagcaactccttctgcagcaacgtgaccggcaactacaggaggggctacgtctgcaggtgcaggaggggctacggcggcaacccatacgtggctggcggctgccaagacatcgacgagtgcaagctcgcgggcaggtgctacggcgagtgcaccaacaccccaggcgactaccagtgcaggtgcccaaggggcgccaggggcgacccgcgcatccccaacggctgcgtgaagaccaacctgggcctctccgtgggcatcggcgtcggcagcggcgctggcctgctcgtgatgggcctgggcgcggccttcctcaagaggaaggtcaagaagcaaagggcccgcatgctgaggcaaaagttcttcaagcagaaccgcggccacctgctccaacagctcgtgtcccagaaggccgacatcgcggagcggatgatcatcccactgtccgagctggagaaggccaccaacaacttcgacaagagcagggagctgggcggcggcggccacggcaccgtctacaagggcatcctgtccgacctccatgtggtcgccatcaagaagtccaaggaggcggtgcaaagggagatcgacgagttcatcaacgaggtcgccatcctcagccagatcaaccaccgcaacgtggtcaagctgttcggctgctgcctggagaccgaggtgccgctgctcgtctacgagttcatctccaacggcaccctgtaccaccatctccacgtggagggcccaatgagcctgccgtgggaggacaggctcaggatcgctaccgagaccgcccgcgcgctggcctacctccattccgcggtgagcttcccaatcatccaccgggacatcaagtcccataacatcctgctcgacggcagcctcaagaccaaggtctccaacttcggcgccagccggtgcatcccagctgagcaaaccggcatcaccaccgtggtccagggcaccctgggctacctcgaccccatgtactactacaccggcaggctgaccgagaagtccgacgtgttcagcttcggcgtggtcctcatcgagctgctcacccgcaagaagccgtactcctacaggagcccagacgacgagtccctcgtcacccacttcaccgccctgctcacccaaggcaacctgggcgacatcctcgacccacaggtgaaggaggaggggggcgaggaggtcaaggagatcgctgtgctggcggtcgcttgcgcgaagctcaaggtggaggagcggcccaccatgaggtga

(19) United States
(12) Patent Application Publication
(10) Pub. No.: US 2013/0269058 A1
(43) Pub. Date: Oct. 10, 2013

(54) PLANTS RESISTANT TO FUNGAL PATHOGENS AND METHODS FOR PRODUCTION THEREOF

(71) Applicants: Jean-Benoit Morel, Montpellier (FR); Amandine Delteil, Montpellier (FR); Francois Jean Georges Torney, Chappes (FR)

(72) Inventors: Jean-Benoit Morel, Montpellier (FR); Amandine Delteil, Montpellier (FR); Francois Jean Georges Torney, Chappes (FR)

(73) Assignee: GENOPLANTE-VALOR, PARIS (FR)

(21) Appl. No.: 13/825,598

(22) PCT Filed: Sep. 23, 2011
(86) PCT No.: PCT/EP11/66573

§ 371 Date: Jun. 4, 2013

(30) Foreign Application Priority Data
Sep. 23, 2010 (EP) 10306022.4

(51) Int. Cl. C12N 15/82; A01N 43/16; C12Q 1/68

(52) U.S. Cl. CPC C12N 15/8282; C12Q 1/6897; A01N 43/16
USPC 800/279; 800/301; 536/23.1; 435/320.1; 435/418; 435/6.13; 504/292

(57) ABSTRACT

The present invention relates to plant genes involved in positive regulation of resistance to fungal pathogens and uses thereof. More particularly, the present invention relates to plants which overexpress WAK91 gene, or an ortholog thereof, and having increased resistance to fungal pathogens. The invention also relates to methods for producing modified plants having increased or improved fungal disease and pathogen resistance. Furthermore, the invention relates to methods of screening and identifying molecules that induce WAK91 gene expression.

### FIELD OF THE INVENTION

The invention relates generally to the field of agricultural biotechnology and plant diseases. In particular, the invention relates to plant genes involved in positive regulation of resistance to fungal pathogens and uses thereof. More specifically, the invention relates to plants overexpressing WAK91 gene, or an ortholog thereof and having increased or improved fungal pathogen resistance. The invention also relates to methods for producing modified plants resistant to fungal diseases. Furthermore, the invention relates to methods of screening and identifying molecules that induce WAK91 gene expression.

### BACKGROUND OF THE INVENTION

To meet the increasing demand on the world food supply, it will be necessary to produce up to 40% more rice by 2030 (Khush, 2005). This will have to be on a reduced sowing area due to urbanization and increasing environmental pollution. For example, the sowing area in China decreased by 8 million hectares between 1996 and 2007. Improvement of yield per plant is not the only way to achieve this goal; reduction of losses by biotic and abiotic stress is also a solution. According to FAO estimates, diseases, insects and weeds cause as much as 25% yield losses annually in cereal crops (Khush, 2005).

Fungal and bacterial pathogens represent a permanent threat on rice cultivation. In particular, fungal diseases can cause important losses (between 1 and 10%) regionally (Savary et al. 2000). In China alone, it is estimated that 1 million hectares are lost annually because of blast disease (Kush and Jena 2009). Between 1987 and 1996, fungicides represented, for example, up to 20 and 30% of the culture costs in China ($46 Million) and Japan ($461 Million) respectively.

Blast disease is caused by the ascomycete Magnaporthe oryzae, also known as rice blast fungus. Members of the M. grisea/M. oryzae complex (containing at least two biological species: M. grisea and M. oryzae) are extremely effective plant pathogens as they can reproduce both sexually and asexually to produce specialized infectious structures known as appressoria that infect aerial tissues and hyphae that can infect root tissues. Magnaporthe fungi can also infect a number of other agriculturally important cereals including wheat, rye, barley, and pearl millet causing diseases called blast disease or blight disease.

Other plant pathogens of economic importance include Fusarium, Thielaviopsis, Verticillium, Rhizoctonia and Puccinia species. Fusarium contamination in cereals (e.g., barley or wheat) can result in head blight disease. For example, the total losses in the US of barley and wheat crops between 1991 and 1996 have been estimated at $3 billion (Brewing Microbiology, 3rd edition. Priest and Campbell, ISBN 0-306-47288-0).

Pathogen infection of crop plants can have a devastating impact on agriculture due to loss of yield and contamination of plants with toxins. Currently, outbreaks of blast disease are controlled by applying expensive and toxic fungicidal chemical treatments using for example probenazole, tricyclazole, pyroquilon and phthalide, or by burning infected crops. These methods are only partially successful since the fungal pathogens are able to develop resistance to chemical treatments.

To reduce the amount of fungicides used, plant breeders and geneticists have also been trying to identify disease resistance loci and exploit the plant's natural defense mechanism against pathogen attack. However, pathogens may mutate and overcome the protection conferred by resistance genes.

Plants can recognize certain pathogens and activate defense in the form of the resistance response that may result in limitation or stopping of pathogen growth. Many resistance (R) genes, which confer resistance to various plant species against a wide range of pathogens, have been identified. However, the key factors that switch these genes on and off during plant defense mechanisms remain poorly understood.

The vast majority of the known R genes code for proteins carrying nucleotide-binding sites and leucine-rich repeat motifs (NBS-LRR) (Jones and Dangl, 2006). Many R genes identified in rice are NBS-LRR genes (Ballini et al. 2008; White and Yang 2009). Most of the products of R genes recognize pathogen effectors developed by pathogens to inhibit defense (e.g. Lee et al. 2009).

After recognition mediated by the R gene, signal transduction occurs causing a deep transcriptional re-programming of the cell (Eulgem 2005) leading to the activation of defense responses per se. These include production of antimicrobial secondary metabolites such as phytoalexins like momilactones in rice (Peters et al., 2006), pathogenesis-related (PR) proteins, e.g., chitinases, glucanases, PBZ1 in rice (Jwa et al., 2006; van Loon et al., 2006), cell-wall strengthening (Hückelhoven 2007) and programmed cell death known as the hypersensitive response (HR) (Greenberg and Yao, 2004). The genes that act downstream of the disease resistance pathway are collectively called defense genes. A disadvantage of most R genes is to be rapidly circumvented by the pathogen.

Pathogen recognition can also occur through the action of plant proteins called PRR (Pattern Recognition Receptor). The pathogen-specific molecules that are recognized by PRRs are called pathogen-associated molecular patterns (PAMPs) and include bacterial carbohydrates (e.g. lipopolysaccharide or LPS, mannose), nucleic acids (e.g., bacterial or viral DNA or RNA), bacterial peptides (e.g., flagellin), peptidoglycans lipotechoic acids, N-formylmethionine, lipoproteins and fungal glucans. However, there are very few data concerning the implication of these PRR receptors in defense mechanisms of plants.

Consequently, there exists a high demand for novel efficient methods for controlling plant diseases such as blast disease, as well as for producing plants of interest with increased resistance to fungal pathogens.

### SUMMARY OF THE INVENTION

The present invention provides novel and efficient methods for producing plants resistant to pathogens. Surprisingly, the inventors have demonstrated that WAK91 is a positive regulator of plant resistance to fungal pathogens. Moreover, the inventors have shown that over-expressing said gene increases plant resistance to fungal disease. The inventors have further shown that WAK91 is highly responsive to chitin, a component found in all fungi, demonstrating the broad utility and advantages of the invention. In addition, the inventors have identified orthologs of WAK91 in various plants, thus extending the application of the invention to different cultures.

An object of this invention therefore relates to cells or plants which overexpress a WAK91 protein or an ortholog thereof.

As will be further disclosed in the present application, the overexpression of WAK91, or an ortholog thereof, may be induced by deletion, insertion and/or substitution of one or more nucleotides, site-specific mutagenesis, ethyl methanesulfonate (EMS) mutagenesis or targeting induced local lesions in genomes (TILLING); induction of the WAK91 gene promoter; or introduction into said plant of an expression cassette comprising a nucleic acid sequence coding for WAK91 or its ortholog.

As will be discussed, the plants of the invention exhibit an increased or improved resistance to fungal pathogens. Preferably, said plants are cereals selected preferably from rice, wheat, barley, oat, rye, sorghum or maize.

The invention also relates to gain-of-function WAK91 mutant cereal plants with increased resistance to fungal pathogens.

A further object of this invention relates to seeds of a plant of the invention.

Another object of this invention relates to plants, or descendents of plants, grown or otherwise derived from said seeds.

A further object of the invention relates to a method for producing a plant having increased resistance to fungal pathogens, wherein the method comprises the following steps:

(a) introducing into a cell of said plant a nucleic acid construct comprising a nucleic acid sequence encoding WAK91, or an ortholog thereof, under the control of a constitutive promoter enabling the expression of said nucleic acid sequence;
(b) optionally, selection of plant cells of step (b) which express WAK91, or an ortholog thereof;
(c) regeneration of plants from cells of step (a) or (b); and
(d) optionally, selection of a plant of (d) with increased resistance to fungal pathogens.

The invention also relates to a method for conferring or increasing resistance to fungal pathogens to a plant, comprising a step of inducing permanently or transiently the expression of WAK91 gene in said plant.

A further object of the invention relates to an isolated cDNA comprising a nucleic acid sequence selected from:
(a) a nucleic acid sequence which encodes WAK91 or an ortholog thereof, or a fragment thereof;
(b) a nucleic acid sequence of SEQ ID NO: 1, 3, 4 or 16, or a fragment thereof;
(c) a nucleic acid sequence which hybridizes to the sequence of (a) or (b) under stringent conditions, and encodes a WAK91 kinase or an ortholog thereof; and
(d) a mutant of a nucleic acid sequence of (a), (b) or (c).

A further object of the invention relates to a nucleic acid molecule comprising the sequence of a WAK91 gene promoter, or of an ortholog thereof, operably linked to a reporter gene.

A further object of the invention relates to a nucleic acid molecule comprising the sequence of a WAK91 gene, or of an ortholog thereof, operably linked to a heterologous promoter, preferably a constitutive promoter.

A further object of the invention relates to recombinant vectors comprising one of the above nucleic acid molecules.

Further objects of the invention relate to cells or plants transformed with such recombinant vectors, and to seeds of the transformed plants.

The invention also relates to a method of identifying a molecule that modulates WAK91 gene expression, the method comprising:

(a) providing a cell comprising a nucleic acid construct that comprises the sequence of a WAK91 gene promoter operably linked to a reporter gene;
(b) contacting the cell with a candidate molecule;
(c) measuring the activity of WAK91 promoter by monitoring of the expression of a marker protein encoded by the reporter gene in the cell;
(d) selecting a molecule that modulates the expression of the marker protein.

Preferably, such a molecule increases the expression of the WAK91 gene promoter.

A further object of the invention relates to the use of a molecule that induces or stimulates WAK91 gene expression for increasing resistance of plants to fungal pathogens. Such a molecule may be identified according to the above method.

The invention is applicable to produce cereals having increased resistance to pathogens, and is particularly suited to produce resistant rice.

### BRIEF DESCRIPTION OF THE DRAWINGS

FIG. 1: WAK91 expression during Magnaporthe oryzae infection. Plants were either inoculated with a virulent isolate (FR13) or an avirulent isolate (CL367). The induction (4×) of the WAK91 gene is observed early (8 hpi, post inoculation) during infection by an avirulent (CL367) and virulent (FR13) isolate, most likely before penetration (developmental phase of the fungus shown below). (A) The expression of the WAK91 gene was monitored using QRT-PCR. The activity of the gene was normalized using the actin control; absolute values are shown for 3 replicates; Mock: non infected plants; (B) The expression value in infected plants was divided by the expression value in non-infected plants and the ratio was log 2 transformed. This experiment was repeated three times and the mean of ratios is shown.

FIG. 2: Chitin induces WAK91 expression. Chitin fragments (two concentrations) were sprayed on healthy rice plants. Chitin alone is sufficient to induce WAK91 (up to 4×) in entire plants after 15 minutes. The PR5 and PDX223 classical defense genes are induced later. (A) The expression value of the WAK91, PR5 and PDX223 gene expression (normalized by actin) was measured at the indicated time after treatment. (B) The expression value after chitin treatment was divided by the expression value in untreated plants and the ratio was log transformed.

FIG. 3: Constructs used for over-expressing the WAK91 gene or for WAK91 promoter analysis.

(A) Vectors for over-expressing the WAK91 gene: the WAK91 coding sequence was cloned into the pCAMBIA2300 OX vector using the BP clonase (Invitrogen). (B) Vectors for promoter WAK91-GUS fusion analysis: the promoter of the WAK91 gene was amplified by PCR on genomic DNA from Nipponbare cultivar and the PCR fragment was digested by BamHI/EcoRI and ligated into pCambia1391-Z. This vector was used to transform rice (cultivar Nipponbare) using a derived protocol from Toki et al. (2006). (C) Vectors for overexpressing the WAK91 gene of Oryza sativa in NB1 wheat line. (D) Construction of SynOsWak91 TaMod plasmid: digestion SapI/ligation of plasmids: pUC19 SapI-12 rubi3, pUC57_BGA_{—}0201_WA, pUC19 SapI-34 SAc66 and pBIOS2028.

FIG. 4: WAK91-overexpressing plants are more resistant to infection by Magnaporthe. (A) Plants overexpressing WAK91 gene (3606-3610 OX-WAK91 TO plants) and plants transfected with empty vector (3647-3650 empty vector (EV) TO plants) were inoculated with the virulent isolate FR13 of Magnaporthe oryzea. Symptoms are shown 5 days after infection. The brown and grey spots represent sporulating lesions. All lines over-expressing the WAK91 gene are more resistant to infection by M. oryzae isolate FR13. (B) Expression of the WAK91 gene in plants overexpressing WAK91(OX) and in plants transfected with empty vector (EV); The expression level of the WAK91 gene was normalized using the actin gene as a reference. (C) The WAK91-overexpressing plants are more resistant to M. oryzae isolate FR13 (number of lesions/unit size).

FIG. 5: WAK91 expression after inoculation with Puccinia triticina (wheat pathogen). The non-adapted fungus Puccinia triticina induces the WAK91 gene. (A) Absolute values of WAK91 expression monitored after inoculation with Puccinia triticina; Mock: non infected plants; (B) The expression value in infected plants was divided by the expression value in non-infected plants and the ratio was log 2 transformed.

FIG. 6: WAK91-defective plants are more susceptible to Magnaporthe. WAK91-defective plants mutated with Tos17 or wild type plants (Wt) were inoculated with the virulent isolate FR13 of Magnaporthe oryzae. It is observed that the WAK91-defective plants are more susceptible to infection by M. oryzae isolate FR13. (A) Disease symptoms in the form of brown and grey spots (lesions) are shown on susceptible WAK91 mutant plants and control wild-type nipponbare plants; (B) The WAK91 gene is no longer expressed in mutant as compared to the sister line not mutated by Tos17 (Wt); (C) The WAK91 Tos17 mutants are more susceptible to M. oryzae isolate FR13 (number of lesions/unit surface). The lesion number is increased by 3-fold.

### DETAILED DESCRIPTION OF THE INVENTION

The WAK family of genes code for proteins belonging to a group of wall-associated kinases (WAK). These kinases contain an extracellular domain containing an EGF-domain of unknown function, a transmembrane domain and a cytoplasmic kinase domain. As indicated in the examples, there is no substantial sequence homology between these WAK genes and it is not possible to predict biological function from sequence data.

The inventors have now discovered that WAK91 is a positive regulator of plant resistance to pathogens, i.e., its presence or over-expression increases resistance. In comparison to other potential positive regulators, the inventors have surprisingly found that the basic expression level of WAK91 in plant cells is very low, so that overexpression thereof can be easily obtained. Furthermore, the inventors have demonstrated that WAK91 is very rapidly induced during infection by a pathogen. As shown in the experimental part, WAK91 is induced within minutes following infection, allowing very rapid defense mechanisms to be engaged. This is the first example of a gene induced early in rice by fungal infection. WAK91, and orthologs thereof, thus represent novel and highly valuable targets for producing plants of interest with increased resistance to fungal pathogens.

The present invention thus relates to methods for increasing pathogen resistance in plants based on a regulation of WAK91. The invention also relates to plants or plant cells which overexpress WAK91 gene, or an ortholog thereof. The invention also relates to constructs (e.g., nucleic acids, vectors, cells, etc) suitable for production of such plants and cells, as well as to methods for producing plant resistance regulators.

The present disclosure will be best understood by reference to the following definitions:

### DEFINITIONS

As used therein, the term “WAK91 protein” designates a wall-associated kinase protein comprising the amino acid sequence of SEQ ID NO: 2 (which corresponds to the WAK91 amino acid sequence of Oryza sativa), and any natural variant thereof (e.g., variants present in other (rice) plants as a result of polymorphism). Within the context of the present invention, the term “WAK91 gene” designates a gene or nucleic acid that codes for a WAK91 wall-associated kinase protein. In particular, a “WAK91 gene” includes any nucleic acid encoding a protein comprising SEQ ID NO: 2, or a natural variant of such a protein. A specific example of a WAK91 gene comprises nucleic acid sequence of SEQ ID NO: 1 (which corresponds to WAK91 nucleotide sequence of Oryza sativa) or nucleic acid sequence of SEQ ID NO: 16 (which corresponds to an optimized OsWAK91 sequence).

Within the context of the present invention, the term “ortholog” designates a related gene or protein from a distinct species, having a level of sequence identity to WAK91 above 50% and a WAK91-like activity. An ortholog of WAK91 is most preferably a gene or protein from a distinct species having a common ancestor with WAK91, acting as a positive regulator of plant resistance, and having a degree of sequence identity with WAK91 superior to 50%. Preferred orthologs of WAK91 have a sequence of at least 60%, preferably at least 63 or 71%, especially preferably at least 75, 80, 90, 95% or more sequence identity to the sequence shown in SEQ ID NO: 1 or 2. WAK91 orthologs can be identified using such tools as “best blast hit” searches or “best blast mutual hit” (BBMH). WAK91 orthologs have been identified by the inventors in various plants, including wheat or sorghum (see Table 2 and sequence listing). Specific examples of such orthologs include the nucleic acid sequence of SEQ ID NO: 3, the nucleic acid sequence of SEQ ID NO: 4 and the amino acid sequence of SEQ ID NO: 5.

Within the context of the present invention, the term “pathogens” designates all pathogens of plants in general. More preferably the pathogens are fungal pathogens. In a particular embodiment, fungal pathogens are cereal fungal pathogens. Examples of such pathogens include, without limitation, Magnaporthe, Puccinia, Aspergillus, Ustilago, Rhizoctonia, Septoria, Erisyphe and Fusarium species. In the most preferred embodiment, the pathogen is Magnaporthe oryzae. The invention is particularly suited to create rice resistant to Magnaporthe.

A promoter is “heterologous” to a gene when said promoter is not naturally associated to said gene. A heterologous promoter may be natural, synthetic, recombinant, hybrid, etc. It may be of cellular or viral origin. The heterologous promoter is preferably a strong and/or constitutive promoter.

Different embodiments of the present invention will now be further described in more details. Each embodiment so defined may be combined with any other embodiment or embodiments unless otherwise indicated. In particular, any feature indicated as being preferred or advantageous may be combined with any other feature or features indicated as being preferred or advantageous.

### WAK91-Overexpressing Plants

As previously described, the present invention is based on the finding that WAK91 gene is a positive regulator of plant resistance to fungal pathogens. The inventors have demonstrated that the overexpression of WAK91 increases plant resistance to fungal pathogens.

Therefore, according to a first embodiment, the invention relates to a plant or a plant cell which overexpresses a WAK91 protein or an ortholog thereof. Preferably, the plant is a cereal. More preferably, the cereal is selected from rice, wheat, sorghum, oat, rye, barley or maize. In the most preferred embodiment the plant is rice, for example Oryza sativa indica, Oryza sativa japonica or Nipponbare. Preferred plants or plant cells of the invention exhibit an increased resistance to pathogens, preferably to fungal pathogens.

In another variant, the invention relates to a plant with increased resistance to fungal pathogens, wherein said increased resistance is due to overexpression of a WAK91 protein, or an ortholog thereof.

In another embodiment, the invention relates to transgenic plants or plant cells which have been engineered to be (more) resistant to fungal pathogens by overexpression of WAK91 or an ortholog thereof. In a particular embodiment, the modified plant is a gain-of-function WAK91 mutant cereal plant, with increased resistance to fungal pathogens.

The invention also relates to a seed of a plant of the invention, as well as to a plant, or a descendent of a plant, grown or otherwise derived from said seed, said plant having an increased resistance to pathogens.

The invention also relates to vegetal material of a plant of the invention, such as roots, leaves, flowers, callus, etc.

Within the context of this invention, the term “overexpressed” or “overexpression”, in relation to WAK91 or its orthologs, indicates an increase in the level of active WAK91 protein present in the cell or plant in comparison with a level of WAK91 expression in a wild type plant. Such an increase is typically of about 2-fold, 3-fold, 4-fold, 5-fold, 10-fold, 100-fold, or more since basal expression of WAK91 is extremely low. The term “overexpression” also designates a modified expression profile of WAK91 or its orthologs, such as a constitutive expression.

Overexpression of WAK91 or orthologs thereof may be obtained by techniques known per se in the art such as, without limitation, by genetic means, enzymatic techniques, chemical methods, or combinations thereof. Overexpression may be conducted at the level of DNA, mRNA or protein, and induce the expression (e.g., transcription or translation) or the activity of WAK91. A preferred overexpression method affects expression and leads to the increased production of a functional WAK91 protein in the cells. It should be noted that the induction of WAK91 may be transient or permanent.

In a first embodiment, overexpression of WAK91 or its orthologs is induced by any mutation in the WAK91 gene or its orthologs, for example point mutation, deletion, insertion and/or substitution of one or more nucleotides in a DNA sequence. This may be performed by techniques known per se in the art, such as e.g., site-specific mutagenesis, ethyl methanesulfonate (EMS) mutagenesis, targeting induced local lesions in genomes (TILLING), homologous recombination, conjugation, etc. A particular approach is gene overexpression by insertion a DNA sequence through transposon mutagenesis using mobile genetic elements called transposons, which may be of natural or artificial origin.

DNA mutations may also be introduced within the WAK91 gene or its orthologs, or within a sequence of the promoter of WAK91 or its orthologs. Mutations in the coding sequence may result in gain of function by increasing biological activity and efficacy of the WAK91 protein. This may be by increasing activation of WAK91 targets via, for example, increased phosphorylation of molecules implicated in the WAK91 kinase signaling pathway. Alternatively, introducing mutations into the promoter sequence may result in gain of function by induction of the promoter activity by controlling and enhancing transcription of the WAK91 gene.

In another particular embodiment, WAK91 overexpression is induced by introduction into a plant of an expression cassette comprising a nucleic acid sequence coding for WAK91 or its ortholog under control of a promoter enabling the expression of said nucleic acid sequence.

WAK91 overproduction in a plant may also be induced by mutating or silencing genes involved in the WAK91 kinase biosynthesis pathway. Alternatively, WAK91 synthesis and/or activity may also be enhanced by inhibiting the expression of negative regulators of WAK91, such as transcription factors or second messengers, using techniques known in the art as described above or by gene silencing using RNA interference etc.

WAK91 overexpression may also be performed transiently, e.g., by applying (e.g., spraying) an exogenous agent to the plant, for example molecules that induce WAK91 expression or activity.

Preferred overexpression is a constitutive expression under control of a constitutive promoter. Such constitutive expression, as illustrated in the examples, leads to a drastic increase in the expression of an active WAK91 protein in the plant, while the plant is still viable.

The invention thus provides a method for producing a plant having increased resistance to pathogens, preferably to fungal pathogens, wherein the method comprises the following steps:
(a) introducing into a cell of said plant a nucleic acid construct comprising a nucleic acid sequence encoding WAK91, or an ortholog thereof, under the control of a promoter, preferably constitutive promoter, enabling the expression of said nucleic acid sequence;
(b) optionally, selection of plant cells of step (b) which express WAK91, or an ortholog thereof;
(c) regeneration of plants from cells of step (a) or (b); and
(d) optionally, selection of a plant of (d) with increased resistance to fungal pathogens.

In a preferred method, the promoter is a heterologous promoter functional in plant cells. Examples of such promoters include, but are not limited to, constitutive promoters, plant tissue-specific promoters, plant development-stage-specific promoters, inducible promoters, viral promoters as well as synthetic or other natural promoters. In a preferred aspect, the promoter is a constitutive or an inducible promoter.

Constitutive promoters may include, for example CaMV 35S promoter (Odell et al. (1985) Nature 313, 9810-812), rice actin promoter (McElroy et al. (1990) Plant Cell 2: 163-171) and ubiquitin promoter (Christian et al. (1989) Plant Mol. Biol. 18 (675-689); pEMU (Last et al. (1991) Theor Appl. Genet. 81: 581-588); MAS (Velten et al. (1984) EMBO J. 3. 2723-2730); ALS promoter (U.S. application Ser. No. 08/409,297), and the like.

A nucleic acid molecule may be introduced into a plant cell by any means, including transfection, transformation, transduction, electroporation, particle bombardment, agroinfection, etc. In a preferred embodiment, a nucleic acid molecule is introduced via Agrobacterium transformation using the Ti plasmid as described e.g., by Toki et al. (2006).

According to the present invention, the introduced nucleic acid molecule may be maintained in the plant cell stably. Alternatively, the introduced nucleic acid molecule may be transiently expressed or transiently active.

Selection of a plant which overexpresses WAK91 can be made by techniques known per se to the skilled person (e.g., PCR, hybridization, use of a selectable marker gene, protein dosing, western blot, etc.).

Plant generation from the modified cells can be obtained using methods known per se to the skilled worker. In particular, it is possible to induce, from callus cultures or other undifferentiated cell biomasses, the formation of shoots and roots. The plantlets thus obtained can be planted out and used for cultivation. Methods for regenerating plants from cells are described, for example, by Fennell et al. (1992) Plant Cell Rep. 11: 567-570; Stoeger et al (1995) Plant Cell Rep. 14: 273-278; Jahne et al. (1994) Theor. Appl. Genet. 89: 525-533.

The resulting plants can be bred and hybridized according to techniques known in the art. Preferably, two or more generations should be grown in order to ensure that the genotype or phenotype is stable and hereditary.

Selection of plants having an increased resistance to a pathogen can be done by applying the pathogen to the plant, determining resistance and comparing to a wild type plant. Within the context of this invention, the term “increased” resistance to pathogen means a resistance superior to that of a control plant such as a wild type plant, to which the method of the invention has not been applied. The “increased” resistance also designates a reduced, weakened or prevented manifestation of the disease symptoms provoked by a pathogen.

The disease symptoms preferably comprise symptoms which directly or indirectly lead to an adverse effect on the quality of the plant, the quantity of the yield, its use for feeding, sowing, growing, harvesting, etc. Such symptoms include for example infection and lesion of a plant or of a part thereof (e.g., different tissues, leaves, flowers, fruits, seeds, roots, shoots), development of pustules and spore beds on the surface of the infected tissue, maceration of the tissue, accumulation of mycotoxins, necroses of the tissue, sporulating lesions of the tissue, colored spots, etc. Preferably, according to the invention, the disease symptoms are reduced by at least 5% or 10% or 15%, more preferably by at least 20% or 30% or 40%, particularly preferably by 50% or 60%, most preferably by 70% or 80% or 90% or more, in comparison with the control plant.

In the most preferred embodiment, the method of the invention is used to produce rice plants which overexpress a WAK91 protein, more preferably Oryza sativa with increased resistance to Magnaporthe oryzae. Examples of such plants, and their capacity to resist to pathogens are disclosed in the experimental section.

### Nucleic Acids Molecules, Expression Cassettes and Vectors

The present invention also relates to nucleic acid molecules suitable for use in the above methods and/or for constructing plants of the invention.

In a particular embodiment, the invention relates to an isolated cDNA comprising a nucleic acid sequence selected from:
(a) a nucleic acid sequence which encodes WAK91 or an ortholog thereof, or a fragment thereof;
(b) a nucleic acid sequence of SEQ ID NO: 1, 3, 4 or 16, or a fragment thereof;
(c) a nucleic acid sequence which hybridizes to the sequence of (a) or (b) under stringent conditions, and encodes a WAK91 kinase or an ortholog thereof; and
(d) a mutant of a nucleic acid sequence of (a), (b) or (c).

Stringent hybridization/washing conditions are well known in the art. For example, nucleic acid hybrids that are stable after washing in 0.1×SSC, 0.1% SDS at 60° C. It is well known in the art that optimal hybridization conditions can be calculated if the sequence of the nucleic acid is known. Typically, hybridization conditions can be determined by the GC content of the nucleic acid subject to hybridization. Typically, hybridization conditions uses 4-6×SSPE (20×SSPE contains Xg NaCl, Xg NaH2PO4 H2O and Xg EDTA dissolved to 1 l and the pH adjusted to 7.4); 5-10×Denhardts solution (50×Denhardts solution contains 5 g Ficoll), 5 g polyvinylpyrrolidone, 5 g bovine serum albumen; X sonicated salmon/herring DNA; 0.1-1.0% s sodium dodecyl sulphate; optionally 40-60% deionised formamide. Hybridization temperature will vary depending on the GC content of the nucleic acid target sequence but will typically be between 42-65° C.

The present invention also relates to a recombinant expression cassette comprising a nucleic acid molecule as described above, operably linked to a promoter or other regulatory elements functional in a plant such as terminator fragments, polyadenylation sequences, enhancer sequences, reporter genes and other sequences as appropriate.

“Operably linked” means joined as part of the same nucleic acid molecule, suitably positioned and oriented for transcription to be initiated from the promoter. A reporter gene operably linked to a promoter is “under transcriptional initiation regulation” of the promoter.

The promoters useful in the expression cassettes of the invention include, but are not limited to, constitutive promoters, plant tissue-specific promoters, plant development-stage-specific promoters, inducible promoters, viral promoters as well as synthetic or other natural promoters. In a preferred aspect, the promoter is a constitutive or an inducible promoter, as described above.

The present invention also relates to a recombinant vector comprising a nucleic acid molecule or a recombinant expression cassette as described above.

In a particular embodiment, the invention relates to a nucleic acid construct comprising an isolated nucleic acid sequence selected from SEQ ID NO: 1, 3, 4 and 16.

Numerous vectors are available for plant transformation, and the nucleic acid molecules, constructs and expression cassettes of the invention may be used in conjunction with any such vectors.

The selection of the suitable vector for uses and methods of the invention will depend upon preferred transformation technique and the target species for transformation. The vector may be a bi-functional expression vector which functions in multiple hosts. Such a recombinant vector may be used for transforming a cell or a plant in order to increase plant resistance to fungal pathogens, or to screen modulators of resistance.

The present invention also relates to transformed cells into which the vectors of the present invention have been inserted and to methods for producing plants having increased resistance to fungal pathogens using such transformed cells.

### Screening of Plant Resistance Modulators

The invention also discloses novel methods of selecting or producing regulators of plant resistance, as well as tools and constructs for use in such methods.

In a particular aspect, the invention relates to a method for screening or identifying a molecule that modulates plant resistance, the method comprising testing whether a candidate compound modulates WAK91 gene expression or activity. The test can be performed in a cell containing a reporter DNA construct cloned under control of WAK91 promoter sequence, or in a cell expressing WAK91.

Preferably, such a method comprises the following steps:
providing a cell comprising a nucleic acid construct that comprises the sequence of a WAK91 gene promoter operably linked to a reporter gene;
contacting the cell with a candidate molecule;
measuring the activity of WAK91 promoter by monitoring of the expression of a marker protein encoded by the reporter gene in the cell; and
selecting a molecule that modulates the expression of the marker protein.

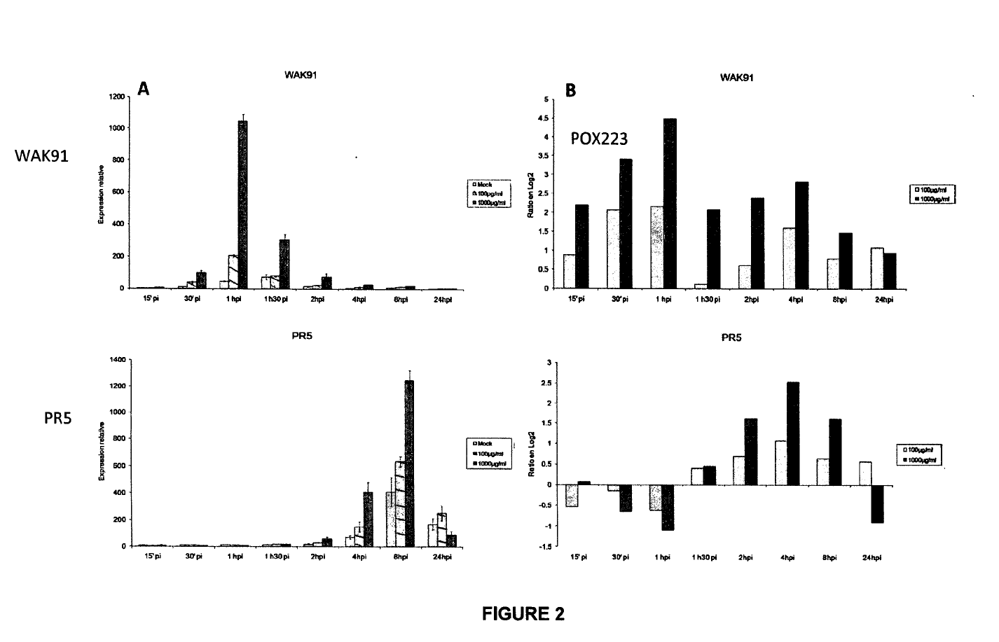

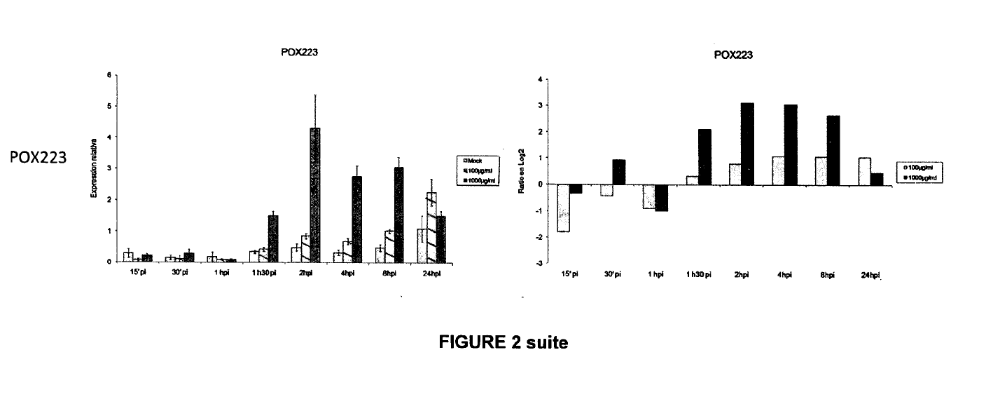

Preferred modulators induce the expression of WAK91. In this regard, the inventors have found that chitin, a major component of fungal cell wall, is involved in WAK91 induction as shown in FIG. 2, showing that small drug or compounds may act to activate WAK91 expression. Thus, in a preferred embodiment, said molecules that modulate the expression of WAK91 are analogs of chitin.

In a further embodiment, the invention thus also relates to the use of a compound that induces WAK91 for increasing resistance of plants to fungal pathogens. Such compounds are typically identified using the above method of screening. The use of such compounds typically comprise exposing a plant to such compound, e.g., by spraying or in admixture with water, thereby causing transient WAK91 overexpression, and transient increase in resistance to pathogens.

In another embodiment, the invention also relates to a nucleic acid construct comprising a promoter sequence of a WAK91 gene or an ortholog thereof, which is operably linked to a reporter gene. In a particular example, the sequence of a WAK91 gene promoter comprises SEQ ID NO: 6 or a functional fragment thereof.

Further aspects and advantages of the invention are provided in the following examples, which are given for purposes of illustration and not by way of limitation.

### EXAMPLES

### Materials and Methods

### Constructs for Over-Expressing the WAK91 Gene

The WAK91 coding sequence was amplified by RT-PCR with primers containing Gateway extensions underlined sequences below)

The primers used were:

ATFL14-F (SEQ ID NO: GGGGACAAGTTTGTACAAAAAAGCAGGCTTCATT CAACCACCGGCTATG and ATFL14-R (SEQ ID NO: 8): GGGGACCACTTTGTAC AAGAAAGCTGGGTCCACACCATGCTCTTGCTGC. The corresponding cDNA (2314 bp including Gateway extensions) was cloned into the pCAMBIA2300 OX vector using the BP clonase (Invitrogen)

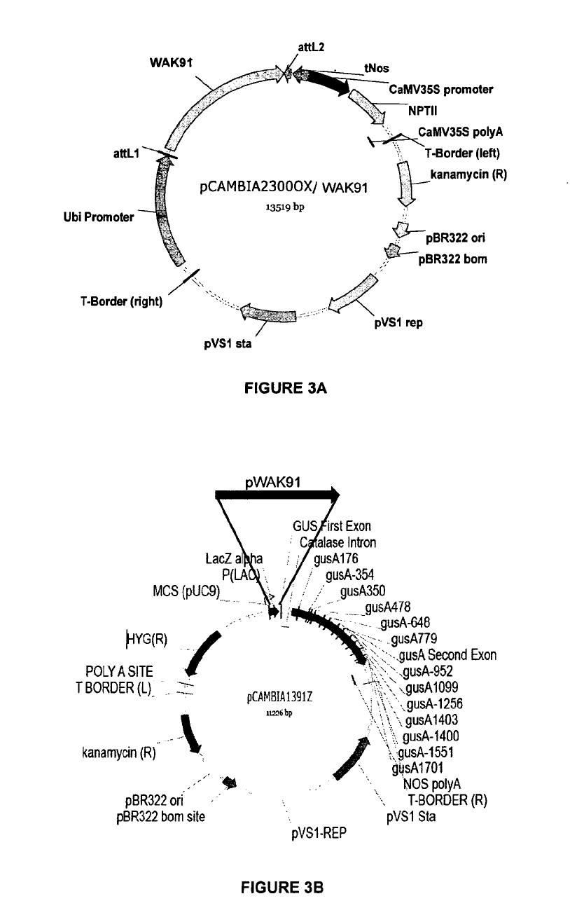

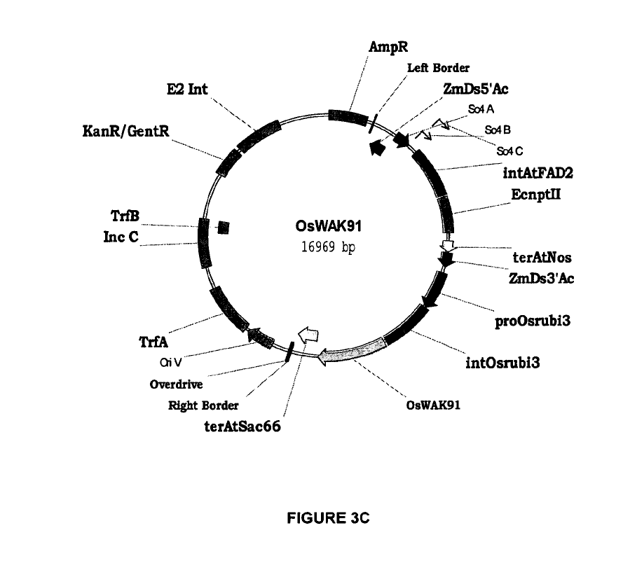

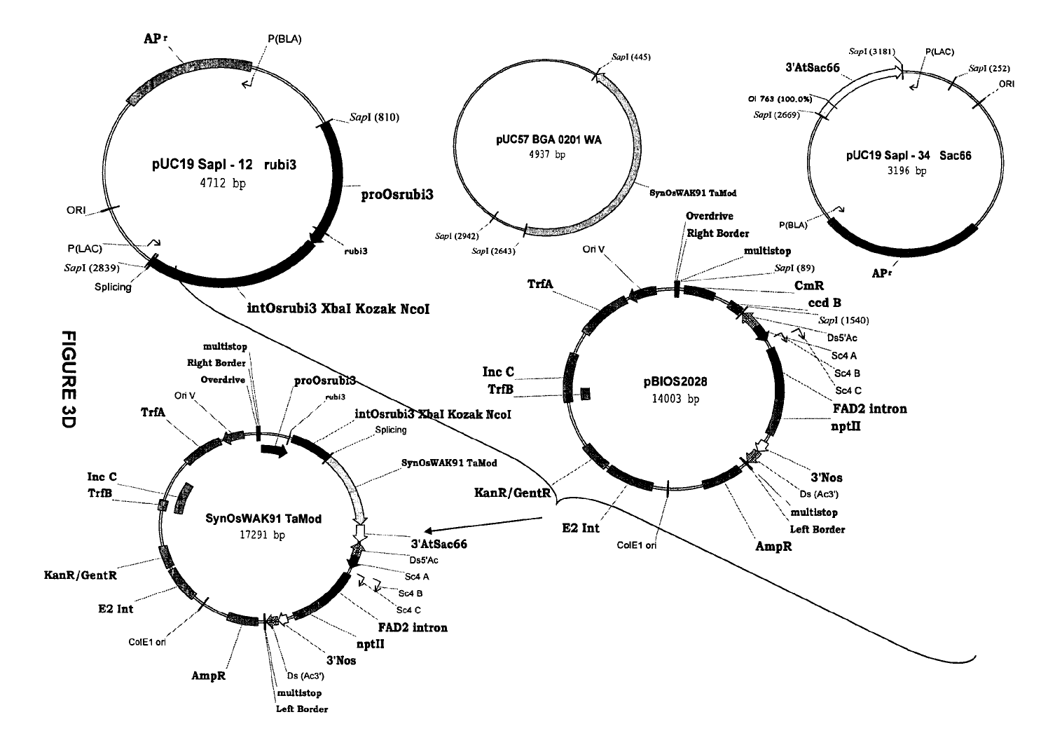

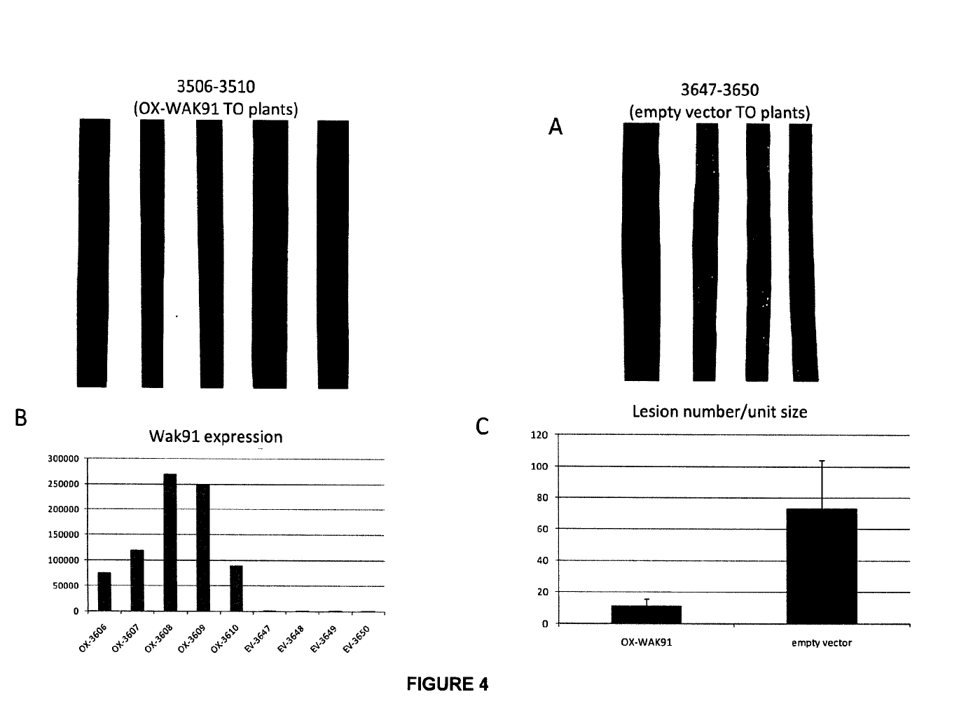

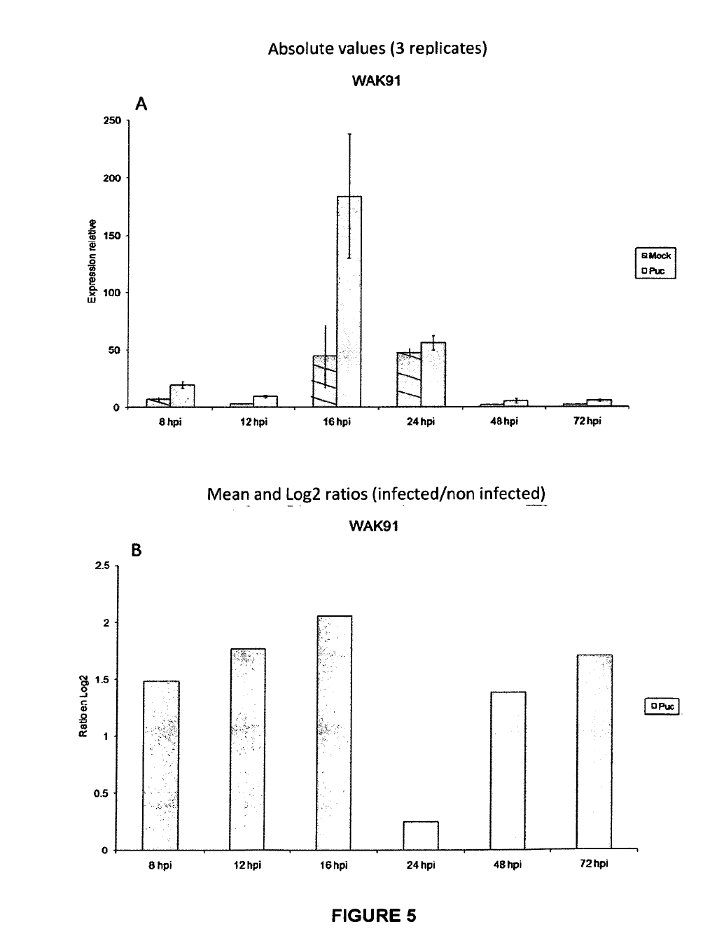

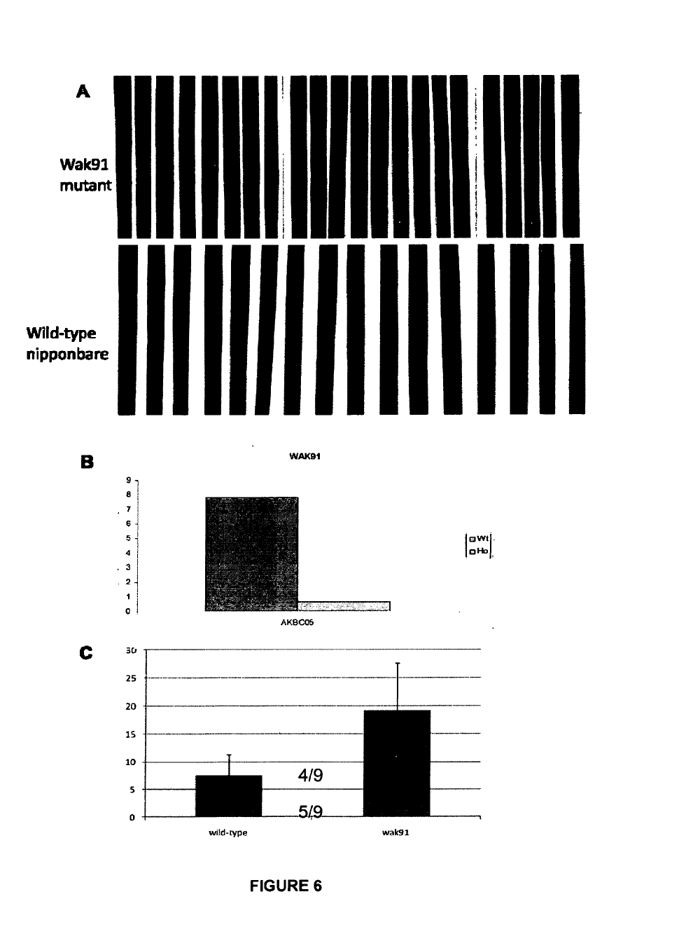

The integrity of the coding sequence was checked by complete re-sequencing of the insert in the transformation vector (pCAMBIA2300 OX/WAK91), as shown in FIG. 3A.

### Rice Transformation

This vector (pCAMBIA2300 OX/WAK91) was used to transform rice (cultivar Nipponbare) using a derived protocol from Toki et al. (2006). Transformant plants (T0) were selected on kanamycin (pCAMBIA2300 OX) or hygromycin (pCAMBIA1391Z). The number of insertions of the T-DNA was measured by Southern Blot using a kanamycin or hygromycin probe. Only single insertion plants were selected for further phenotyping in the T1 generation.

### Vectors for Promoter WAK91-GUS Fusion Analysis

The promoter of the WAK91 gene was amplified by PCR on genomic DNA from Nipponbare cultivar using primers ATp4-F (SEQ ID NO: 9) (ATCTAGGATCCACCGA CTATGAGACAGGTGG) and ATp4-R (SEQ ID NO: 10) (AGTACGAATTCCATAGC CGGTGGTTGAATGA). The PCR fragment was digested by BamHI/EcoRI (sequences included in primers) and ligated into pCambia1391-Z (FIG. 3B).

### WAK91 Mutant Production

The insertion mutant for the WAK91 gene was characterized. This mutant harbors an insertion of the retrotranspososon Tos17 at the genomic position 22317131 of chromosome 9 (WAK91 gene is between 22315245 and 22318384).

Different PCR primers were used to genotype the line at the insertion site. The Tail6 primer (GTACTGTATAGTTGGCCCATGTCC) (SEQ ID NO: 11) is positioned on the Tos17 insertion and the primers AT23F (GCACCAACTGCTCTGTTTCA) (SEQ ID NO: 12) and AT23R (GCAATGGCACTTCTGTCTCA) (SEQ ID NO: 13) are on genomic DNA. A combination of the Tail6 and AT23F primers allows the identification of mutant alleles. The combination of the AT23F and AT23R allows the identification of the wild-type allele.

### Gene Expression Studies

Gene expression was performed using Quantitative RT-PCR as described in Vergne et al (2007). For RT-QPCR applications, frozen tissue were ground in liquid nitrogen. Approximately 500 μl of powder was then treated with 1 ml of TRIZOL supplied by Invitrogen, vortexed for 30 s and incubated at room temperature for 15 minutes. The samples were centrifuged (10 min, 12 000 rpm at 4° C.) and the supernatants were collected in new 2 ml fresh tubes. Then 200 μl of chloroform were added and the samples were shaked for 15 s (no vortex) and incubated at room temperature 5-10 min. After centrifugation (12 000 rpm, 4° C.) 3 phases were obtained. The supernatants (approximatively 400 μl) were transferred to new 1.5 ml fresh tubes, and 200 μl of isopropanol added. Samples were incubated 5 min at room temperature, and then centrifuged (30 min, 10 000 rpm, 4° C.) to obtain RNA pellets. After elimination of isopropanol, pellets were washed with 70% ethanol and resuspendend in distilled water. Polysaccharides were removed by adding a last centrifugation step (10 000 rpm, 4° C., 60 s). They formed a translucent pellet (or drop). RNA samples (5 μg) were denaturated for 5 min at 65° C. with oligo dT (3.5 μM) and dNTP (1.5 μM). They were then subjected to reverse transcription for 60 min at 37° C. with 200 U of reverse transcriptase M-MLV (Promega, Madison, Wis., USA) in the appropriate buffer. Two microlitres of cDNA (dilution 1/10) were then used for quantitative RT-PCR. Quantitative RT-PCR mixtures contained PCR buffer, dNTP (0.25 mM), MgCl2 (2.5 mM), forward and reverse primers (150 or 300 μM), 1 U of HotGoldStar polymerase and SYBR Green PCR mix as per the manufacturer's recommendations (Eurogentec, Seraing, Belgium). Amplification was performed as follows: 95° C. for 10 min; 40 cycles of 95° C. for 15 s, 62° C. for 1 min and 72° C. for 30 s; then 95° C. for 1 min and 55° C. for 30 s. The quantitative RT-PCR (QRT-PCR) reactions were performed using a MX3000P machine (Stratagene) and data were extracted using the MX3000P software. The amount of plant RNA in each sample was normalized using actin (Os03g50890) as internal control. Primers used for the WAK91 gene were ATq41F (TTGCAAGCATGACAGCGGTTAC; SEQ ID NO: 14) and ATq41R (AACCCTCTTCAAGGCCAAACGG; SEQ ID NO: 15).

Treatments with Pathogens

Magnaporthe oryzae was inoculated as described in Vergne et al (2007). The rice cultivar Nipponbare (Oryza sativa L.) and two races, FR13 and CL3.6.7 of blast fungus (Magnaporthe oryzae) were used. The race CL367 is incompatible and race FR13 is compatible with Nipponbare. Rice plants and fungus were grown as described in Berruyer et al (2003). The inoculation was carried out by spraying conidial suspension (2×10⁵ conidia/ml) and mock suspension on the third leaf of three week old rice seedlings. The third leaves were harvested at 0.25, 0.50, 1, 1.5, 2, 4, 8, 24, 48, 72 and 96 h after infection for total RNA extractions and expression analysis by QRT-PCR.

For mutant phenotyping inoculation was carried out by spraying 25×10³ conidia/ml of FR13 race (compatible strain which normally leads to disease) whereas for expression analysis 2×10⁵ conidia/ml of conidial suspension was used, on fourth leaves of four week old plants. All treated seedlings were placed in dark boxes with 100% relative humidity for 24 h. The fourth leaves for mutant phenotyping were harvested and scanned at 5 days after infection for lesion observations and quantifications. Puccinia was inoculated as described in Tufan et al (2009).

### Example 1

### The WAK91 Gene is Induced During Infection by Magnaporthe oryzae

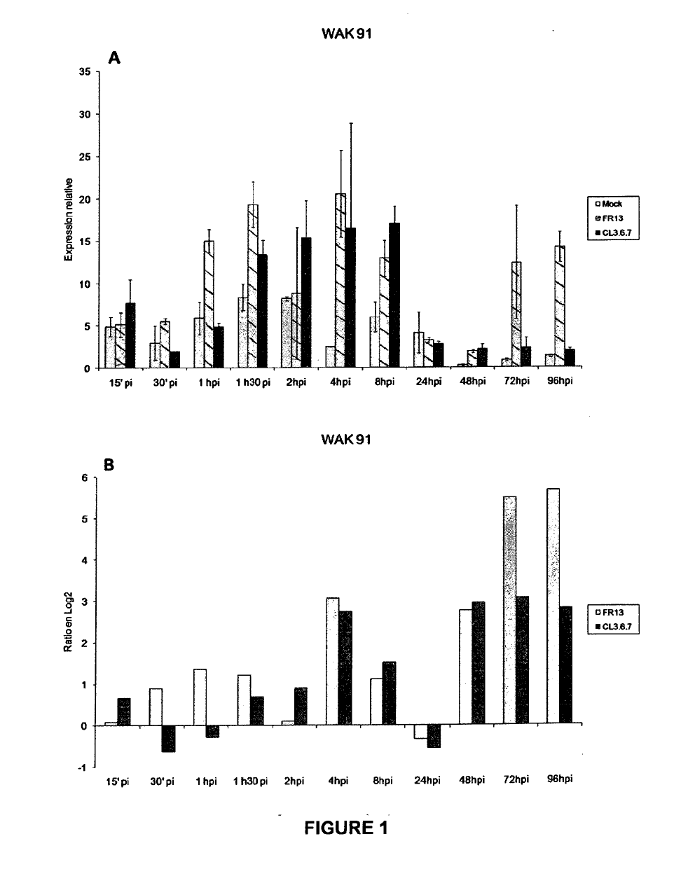

The inventors have observed that the induction of WAK91 gene is observed early during infection by virulent and avirulent isolates of fungal pathogens (FIG. 1). The WAK91 gene is induced up to 4-fold after inoculation by virulent (FR13) and avirulent (CL367) isolates.

### Example 2

### Chitin is Sufficient to Trigger WAK91 Expression

We tested whether this induction pattern could be triggered by chitin. Chitin was purchased at Yaizu Suisankagaku Industrial (Shizuoka, Japan) and used as described in Miya et al (2007). The data show that chitin is sufficient to cause WAK91 induction (FIG. 2).

### Example 3

### WAK91-Overexpressing Plants

I—WAK91-overexpressing lines (OX-3606-3610) were produced and tested. Then, the inventors have verified that the expression of the WAK91 gene was increased in WAK91-overexpressing lines in comparison to lines transfected with empty vectors (EV-3647-3650) (FIGS. 3A, 3B and 4B).

II—OsWAK91-overexpressing wheat lines were produced and tested. The vectors used for overexpressing the WAK91 gene of Oryza sativa in NB 1 wheat line are shown in FIGS. 3C and 3D. For construction of the final SynOsWak91 TaMod plasmid, four following plasmides were mixed, and subjected to one-shot digestion by SapI/ligation:
PUC19rubi3 (comprising the promoter of the rice ubiquitine and its intron enhancer of expression); in an alternative way, the selected combination promoter-intron can be for example from corn ubiquitine or from rice actin;
pUC57 BGA comprising the OsWAK91 optimized sequence of SEQ ID NO: 16);
pBIOS comprising the cassette including nptII selection marker; and
pUC19Sac66 comprising the terminator sequence.

The protocol of wheat transformation was essentially similar to that described previously in WO 00/63398. Wheat tillers, approximately 14 days post-anthesis (embryos approximately 1 mm in length) were harvested from glasshouse grown plants to include 50 cm tiller stem, (22/15° C. day/night temperature, with supplemented light to give a 16 hour day). All leaves were then removed except the flag leaf and the flag leaf was cleaned to remove contaminating fungal spores. The glumes of each spikelet and the lemma from the first two florets were then carefully removed to expose the immature seed. Only these two seed in each spikelet were generally uncovered. This procedure was carried out along the entire length of the inflorescence. The ears were then sprayed with 70% IMS as a brief surface sterilization.

Agrobacterium tumefaciens strains containing the vector for transformation were grown on solidified YEP media with 20 mg/l kanamycin sulphate at 27° C. for 2 days. Bacteria were then collected and re-suspended in TSIM1 (MS media with 100 mg/l myo-inositol, 10 g/l glucose, 50 mg/l IVIES buffer pH5.5) containing 400 μM acetosyringone to an optical density of 2.4 at 650 nm.

Agrobacterium suspension (1 μl) was inoculated into the immature seed approximately at the position of the scutellum: endosperm interface, using a 10 μl Hamilton, so that all exposed seed were inoculated. Tillers were then placed in water, covered with a translucent plastic bag to prevent seed dehydration, and placed in a lit incubator for 3 days at 23° C., 16 hr day, 45 μm-2s-1 PAR.

After 3 days of co-cultivation, inoculated immature seed were removed and surface sterilized (30 seconds in 70% ethanol, then 20 minutes in 20% Domestos, followed by thorough washing in sterile distilled water). Immature embryos were aseptically isolated and placed on W4 medium (MS with 20 g/l sucrose, 2 mg/l 2,4-D, 500 mg/l Glutamine, 100 mg/l Casein hydrolysate, 150 mg/l Timentin, pH5.8, solidified with 6 g/l agarose) and with the scutellum uppermost. Cultures were placed at 25° C. in the light (16 hour day). After 12 days cultivation on W4, embryogenic calli were transferred to W425G media (W4 with 25 mg/l Geneticin (G418)). Calli were maintained on this media for 2 weeks and then each callus was divided into 2 mm pieces and re-plated onto W425G

After a further 2 weeks culture, all tissue was assessed for development of embryogenic callus: any callus showing signs of continued development after 4 weeks on selection was transferred to regeneration media MRM 2K 25G (MS with 20 g/l sucrose, 2 mg/l Kinetin, 25 mg/l Geneticin (G418), pH5.8, solidified with 6 g/l agarose). Shoots were regenerated within 4 weeks on this media and then transferred to MS20 (MS with 20 g/l sucrose, pH5.8, solidified with 7 g/l agar) for shoot elongation and rooting.

The presence of the T-DNA, and the number of copies are quantified by Quantitative PCR.

The transgenic wheat plants overexpressing OsWAK91 are then tested for their resistance to different pathogens, namely Fusarium graminearum and M. oryzae.

### Example 4

### WAK91-Overexpressing Plants are More Resistant to Infection

WAK91-overexpressing lines (OX-3606-3610) lines have been tested for resistance to Magnaporthe virulent isolate FR13 (FIGS. 4A and 4C). Repeated experiments showed that the WAK91-overexpressing mutant plants displayed less lesions than control lines (EV-3647-3650). The average lesion number was 4-fold higher in “EV” plants than in “OX” plants. This indicates that overexpression of the WAK91 gene increases resistance to rice blast.

### Example 5

### The WAK91 Gene is Induced after Infection with Puccinia triticina (Wheat Pathogen)

The non-adapted fungus Puccinia triticina has been shown to induce the WAK91 gene (FIG. 5). The WAK91 gene is induced up to 4-fold after inoculation by Puccinia triticina isolate.

### Example 6

### WAK91-Defective Plants Gene are More Susceptible to Magnaporthe

In order to further demonstrate that the WAK91 is a positive regulator of resistance to Magnaporthe, the inventors have built WAK91-defective plants. Nipponbare plants were mutated by Tos17. FIG. 6 shows that loss of function of the WAK91 gene increases susceptibility to Magnaporthe.

These data thus confirm that the WAK91 is a positive regulator of resistance to Magnaporthe and that the increased resistance to Magnaporthe observed in WAK-overexpressing mutants (FIG. 4) is due to the mutation “gain of function” in the WAK91 gene.

### CONCLUSIONS

Altogether, the expression data and the phenotypical data indicate that the WAK91 gene is a positive regulator of resistance to Magnaporthe.

### Example 7

### WAK91 Homology with Other Genes

There exists a large number of WAK proteins in plant genomes. For example, in rice there is more than 140 WAK proteins, Zhang et al., 2005. However, there is a clear phylogenetic separation between WAK proteins in different plants, and therefore it is not possible to predict the biological function of different WAKs from the available data.

WAK91 is weakly similar to known WAK sequence. For example, the amino acid homology between WAK91 of rice and WAK1 of rice is only 31%. The homology between the WAK91 gene of rice and the RFO1 protein of Arabidopsis is 37% (Table 1).

TABLE 1
Protein homologies between WAK91 and other WAKs

| Name | Accession | AA id RFO1 | AA id WAK1 |
|---|---|---|---|
| wak91 | Os09g38850 | 37% | 31% |

### Example 9

### WAK91 Orthologs

Furthermore, the inventors have carried out Tblastn searches with the WAK91 protein from rice and have identified several orthologs, e.g., in wheat and sorghum. Table 2 shows BBMH established by blasting back (protein or nucleic acid) on rice. To see if homology uncovers phylogenetic relationship and possibly functional homology, the inventors have tested whether the cereal homologs were in turn the best blast hit (Best Blast Mutual Hit=BBMH) on rice.

TABLE 2
Orthologs of WAK91

| Species | SEQ ID NO | Best tblastn | BBMH | % id |
|---|---|---|---|---|
| Wheat | SEQ ID NO: 3 | DQ177499.1 | yes | 71% |
| (WAKL2) | | | | |
| sorghum | SEQ ID NO: 5 | XM_002455661.1 | yes | 63% |

### REFERENCES

Khush, G. (2005) What it will take to Feed 5.0 Billion Rice consumers in 2030. Plant Molecular Biology, 59(1): 1-6.
Khush, G S. and Jena, K K., (2009) Current status and future prospects for research on blast resistance in rice (Oryza sativa L.) In G L Wang (ed.) and B Valent (ed.). Advances in Genetics, Genomics and Control of Rice Blast Disease, p. 1-10, Springer.
Savary, S., Willocquet, L., Elazegui, F. A., Castilla, N. P., and Teng, P. S. (2000) Rice Pest Constraints in Tropical Asia: Quantification of Yield Losses Due to Rice Pests in a Range of Production Situations. Plant Disease, 84(3): 357-369.
Jones, J. D. G., and Dangl, J. L. (2006) The plant immune system. Nature, 444(7117): 323-329.
Ballini, E., Morel, J.-B., Droc, G. t., Price, A., Courtois, B., Notteghem, J.-L., and Tharreau, D. (2008) A Genome-Wide Meta-Analysis of Rice Blast Resistance Genes and Quantitative Trait Loci Provides New Insights into Partial and Complete Resistance. Molecular Plant-Microbe Interactions, 21(7): 859-868.
White, F. F. and Yang, B. (2009) Host and Pathogen Factors Controlling the Rice-Xanthomonas oryzae Interaction. Plant Physiology, 150(4): 1677-1686.
Greenberg, J. T., and Yao, N. (2004) The role and regulation of programmed cell death in plant-pathogen interactions. Cellular Microbiology, 6(3): 201-211.

Hückelhoven, R. (2007) Cell Wall Associated Mechanisms of Disease Resistance and Susceptibility. Annual Review of Phytopathology, 45(1): 101-127.
Jwa, N.-S., Agrawal, G. K., Tamogami, S., Yonekura, M., Han, 0., Iwahashi, H., and Rakwal, R. Role of defense/stress-related marker genes, proteins and secondary metabolites in defining rice self-defense mechanisms. Plant Physiology and Biochemistry, 44(5-6): 261-273.
Lee, S W, Han, S W, Sririyanum, M, Park, C J, Seo, Y S, Ronald, P C. (2009) A type I-secreted, sulfated peptide triggers XA21-mediated innate immunity. Science, 326(5954):850-853.
Peters, R. J. (2006) Uncovering the complex metabolic network underlying diterpenoid phytoalexin biosynthesis in rice and other cereal crop plants. Phytochemistry, 67(21): 2307-2317.
van Loon, L. C., Rep, M., and Pieterse, C. M. J. (2006) Significance of Inducible Defense-related Proteins in Infected Plants. Annual Review of Phytopathology, 44(1): 135-162.
Eulgem T. (2005) Regulation of the Arabidopsis defense transcriptome. Trends Plant Sci. 2005 February; 10(2):71-8. Review.
Vergne E, Ballini E, Marques S, Sidi Mammar B, Droc G, Gaillard S, Bourot S, DeRose R, Tharreau D, Nottéghem J L, Lebrun M H, Morel J B. Early and specific gene expression triggered by rice resistance gene Pi33 in response to infection by ACE1 avirulent blast fungus. New Phytol. 2007; 174(1):159-71. PubMed PMID:17335506.
Tufan H A, McGrann G R, Magusin A, Morel J B, Miché L, Boyd L A. Wheat blast: histopathology and transcriptome reprogramming in response to adapted and nonadapted Magnaporthe isolates. New Phytol. 2009 October; 184(2):473-84. Epub 2009; July 23. PubMed PMID: 19645735.
Berruyer R, Adreit H, Milazzo J, Gaillard S, Berger A, Dioh W, Lebrun M H, Tharreau D. Identification and fine mapping of Pi33, the rice resistance gene corresponding to the Magnaporthe grisea avirulence gene ACE1. Theor Appl Genet. 2003 October; 107(6):1139-47. Epub 2003 June 28. PubMed PMID: 12838393.
Miya A, Albert P, Shinya T, Desaki Y, Ichimura K, Shirasu K, Narusaka Y, Kawakami N, Kaku H, Shibuya N. CERK1, a LysM receptor kinase, is essential for chitin elicitor signaling in Arabidopsis. Proc Natl Acad Sci USA. 2007 Dec. 4; 104(49):19613-8. Epub 2007 November 27. PubMed PMID: 18042724; PubMed Central PMCID: PMC2148337.
Toki S, Hara N, Ono K, Onodera H, Tagiri A, Oka S, Tanaka H. Early infection of scutellum tissue with Agrobacterium allows high-speed transformation of rice. Plant J. 2006 September; 47(6):969-76. PubMed PMID: 16961734.

1-24. (canceled)

25. A plant which overexpresses a WAK91 protein, or an ortholog thereof, as a result of induction of the promoter of the WAK91 gene, or an ortholog thereof, in cells of said plant, or of introduction into cells of said plant of an expression cassette comprising a nucleic acid sequence coding for WAK91, or an ortholog thereof, and wherein said plant exhibits an increased resistance to fungal pathogens.

26. The plant of claim 25, wherein said plant is a cereal plant.

27. The plant of claim 26, wherein said cereal plant is selected from rice, wheat, sorghum, oat, rye, barley or maize.

28. The plant of claim 25, wherein said plant is transformed with a nucleic acid construct comprising an isolated nucleic acid sequence selected from SEQ ID NO: 1, 3, 4 or 16.

29. A seed of the plant of claim 25.

30. A plant, or a descendent of a plant grown or otherwise derived from the seed of claim 29.

31. A method for producing a plant having increased resistance to fungal pathogens, wherein the method comprises:
(a) introducing into a cell of said plant a nucleic acid construct comprising a nucleic acid sequence encoding WAK91, or an ortholog thereof, under the control of a constitutive promoter enabling the expression of said nucleic acid sequence;
(b) optionally, selection of plant cells of step (b) which express WAK91, or an ortholog thereof;
(c) regeneration of plants from cells of step (a) or (b); and
(d) optionally, selection of a plant of (d) with increased resistance to fungal pathogens.

32. The method of claim 31, wherein the plant is a cereal plant.

33. The method of claim 32, wherein said cereal plant is selected from rice, wheat, barley, oat, rye, sorghum or maize.

34. The method of claim 31, wherein said fungal pathogens are selected from Magnaporthe, Puccinia, Aspergillus, Ustilago, Rhizoctonia, Septoria, Erisyphe or Fusarium species.

35. The method of claim 34, wherein said fungal pathogen is Magnaporthe oryzae.

36. A recombinant expression cassette comprising a nucleic acid sequence operably linked to a promoter functional in a plant, said nucleic acid sequence being selected from:
(a) a nucleic acid sequence which encodes WAK91 or an ortholog thereof, or a fragment thereof;
(b) a nucleic acid sequence of SEQ ID NO: 1, 3, 4 or 16, or a fragment thereof;
(c) a nucleic acid sequence which hybridizes to the sequence of (a) or (b) under stringent conditions, and encodes a WAK91 kinase or an ortholog thereof; or
(d) a mutant of a nucleic acid sequence of (a), (b) or (c).

37. The recombinant expression cassette of claim 36, wherein the promoter is a constitutive plant promoter, a plant tissue-specific promoter, a plant development-stage-specific promoter, an inducible promoters or a viral promoter.

38. A recombinant vector comprising the expression cassette of claim 36.

39. A cell or plant transformed with the recombinant vector of claim 38.

40. Seeds of the plant of claim 39.

41. A method of identifying a molecule that modulates WAK91 gene expression, the method comprising:
(a) providing a cell comprising a nucleic acid construct that comprises a WAK91 gene promoter that is operably linked to a reporter gene;
(b) contacting the cell with a candidate molecule;
(c) measuring the activity of WAK91 promoter by monitoring of the expression of a marker protein encoded by the reporter gene in the cell;
(d) selecting a molecule that modulates the expression of the marker protein.

42. The method of claim 41, wherein said molecule increases WAK91 gene expression.

43. A nucleic acid molecule comprising a promoter sequence derived from a WAK91 gene or an ortholog thereof, which is operably linked to a reporter gene.

44. A method for conferring or increasing resistance to fungal pathogens to a plant, comprising a step of inducing or stimulating the expression of WAK91 gene in said plant.

45. The method of claim 44, wherein said method comprises applying chitin to said plant.
